(19) United States
(12) Patent Application Publication
(10) Pub. No.: US 2022/0017460 A1
(43) Pub. Date: Jan. 20, 2022

(54) Sulfonation of Vinylidene Olefins

(71) Applicant: ExxonMobil Chemical Patents Inc., Baytown, TX (US)

(72) Inventors: Mika L. Shiramizu, Houston, TX (US); Alan A. Galuska, Ellijay, GA (US); Jennifer A. Carvajal Diaz, Spring, TX (US)

(21) Appl. No.: 17/296,155

(22) PCT Filed: Nov. 20, 2019
(86) PCT No.: PCT/US2019/062316

§ 371 Date: May 21, 2021

Related U.S. Application Data
(60) Provisional application No. 62/781,635, filed on Dec. 19, 2018.

(30) Foreign Application Priority Data
Jan. 25, 2019 (EP) 19153651.5

(51) Int. Cl. C07C 315/04

(52) U.S. Cl. CPC C07C 315/04

(57) ABSTRACT

Sulfonated reaction products formed from internal vinylidene olefins may display surfactant properties differing from those of the corresponding reaction products formed from sulfonation of alpha olefins and internal olefins having a like number of carbon atoms. Methods for sulfonating internal vinylidene olefins may comprise: providing an internal vinylidene olefin comprising a vinylidene group; exposing the internal vinylidene olefin to a sulfonating reagent; and reacting the sulfonating reagent with the internal vinylidene olefin to form a sulfonated reaction product comprising at least one sulfonated compound formed from the internal vinylidene olefin. Suitable sulfonation conditions may include sulfur trioxide as a sulfonating reagent under thin film or falling film conditions.

### CROSS-REFERENCE TO RELATED APPLICATIONS

This application claims the benefit of Provisional Application No. 62/781,635, filed Dec. 19, 2018 and European Application No. 19153651.5, filed Jan. 25, 2019, the disclosures of which are incorporated herein by reference.

### FIELD

The present disclosure relates to sulfonation of vinylidene olefins and reaction products obtained from the same.

### BACKGROUND

Alpha olefins and internal olefins may undergo sulfonation to form one or more sulfonated reaction products having surfactant properties. Sulfonation of these types of olefins may be accomplished using sulfur trioxide as a sulfonating reagent and thin film contacting conditions, such as falling film conditions. Illustrative falling film sulfonation conditions are described in U.S. Pat. No. 3,169,142.

Several reaction products may be formed when sulfonating alpha olefins and internal olefins. A mixture of hydroxyalkanesulfonates and alkenesulfonates is usually produced, as elaborated further below. The product types and distribution ratios may differ significantly depending on the location of the double bond. The product types and distribution ratios may be further dictated by the amount of sulfonating reagent used with respect to the olefin, the contact time of the sulfonating reagent with the olefin, and the time over which the reaction product is aged. In some instances, there can be significant property differences between the sulfonated reaction products obtained from alpha olefins and internal olefins. As one non-limiting example, the sulfonated reaction products obtained from internal olefins may be particularly suitable for use as surfactants in enhanced oil recovery operations. In addition, the types and distribution ratios of the sulfonated reaction products that are formed may impact their suitability for use in various applications. Formation of colored impurities and non-sulfonated byproducts, for example, may render the sulfonated reaction products unsuitable for use in certain commercial applications.

Scheme 1 below shows various ways in which alpha olefins may react to form sulfonated reaction products. As defined herein, alpha olefins have a terminal alkene functionality appended to only a single hydrocarbyl group at C-2 of the alkene functionality. No additional substitution is present upon the alkene carbon atoms. Although not depicted in Scheme 1, additional chain branching within the hydrocarbyl group may be present in some cases. In a first reaction pathway, alpha olefins bearing an allylic hydrogen atom may react with sulfur trioxide to place a sulfonate group on the terminal carbon atom concurrently with double bond migration to an internal position. The reaction may be considered an ene-type reaction. The resulting internal olefin bearing a terminal sulfonate group is the major product obtained from sulfonation of alpha olefins, although this product may also be produced through competing reaction pathways (e.g., via dehydration). In another reaction pathway for alpha olefins, sulfur trioxide may also react directly with the double bond to form a β-sultone. The initially formed β-sultone may undergo rearrangement upon aging to form a γ-sultone or a δ-sultone. The sultones are typically hydrolyzed collectively in situ with an alkali metal base (e.g., sodium hydroxide) to form hydroxylated compounds, each bearing a terminal sulfonate group and a hydroxyl group at differing internal carbon atoms depending on which sultone isomer was hydrolyzed. For alpha olefins, the hydroxylated compounds formed from γ- and δ-sultones represent a significant fraction of the reaction product (>20%), whereas the hydroxylated compounds formed from β-sultones are typically present in only negligible quantities (<1%).

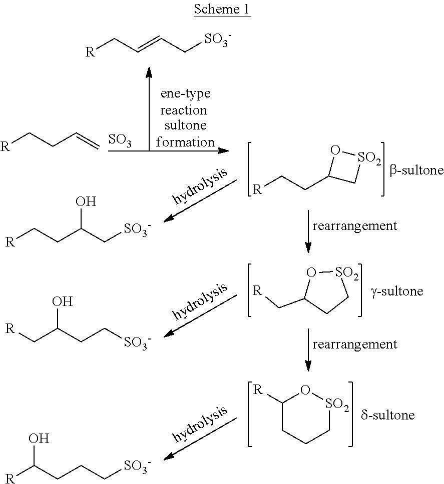

Although not shown in Scheme 1, dehydration of the hydroxylated compounds (formed from the sultone isomers) may also occur to form various internal olefins, each bearing a terminal sulfonate group. One or more of the dehydration products may have a structure corresponding to that formed in the ene-type reaction. In still further reaction pathways (typically minor), any of the sulfonated compounds bearing a double bond may undergo additional sulfonation to form bis-sulfonate compounds, and β-sultones may undergo to additional SO₃ insertion to form pyrosultones. Thus, the reaction mixture obtained from sulfonation of alpha olefins can be exceedingly complex. Variance in the complex product distribution may result in considerable impacts to various physical properties, such as surfactant properties.

Scheme 2 below shows various ways in which internal olefins may react to form sulfonated reaction products. As defined herein, internal olefins bear an alkene functionality at a non-terminal position, and each carbon atom of the alkene functionality may be substituted with one or two hydrocarbyl groups. Any geometric isomer may be present. Again, it is to be appreciated that branched chain internal olefins may undergo similar types of reactions. Since the reactions depicted in Scheme 2 bear considerable similarity to those shown in Scheme 1, the reactions themselves are not described in detail again here. Instead, substantive differences typically seen between the sulfonation of alpha olefins and internal olefins are described hereinafter.

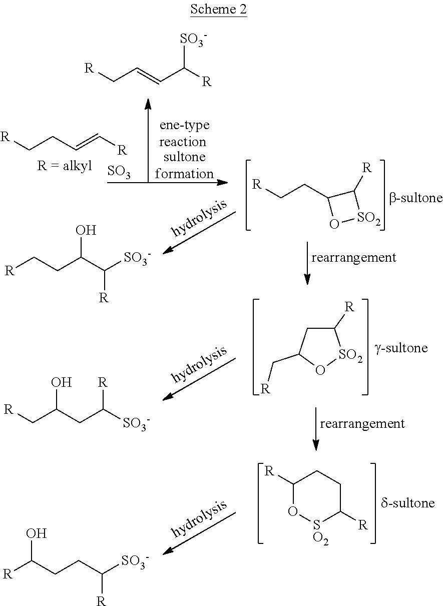

The reaction products obtained from sulfonation of internal olefins may be similar to those obtained from sulfonation of alpha olefins, with the exception of the sulfonate group being located upon an internal carbon atom and a different product distribution resulting. Sulfonation of internal olefins may be further complicated by several additional factors, however. First, different regioisomers are possible when sulfonating an internal olefin, depending upon which carbon atom or side of the double bond is acted upon by the sulfur trioxide. Only one set of possible regioisomers is shown in Scheme 2. Second, the double bond of internal olefins is typically much less reactive than is the terminal double bond in alpha olefins, which may impact the product distribution still further. Namely, due to the lower double bond reactivity, substantial quantities of unreacted internal olefins may remain as a ‘free oil’ following sulfonation, which may impact suitability of the sulfonated reaction products for use in surfactant applications.

The innate structural differences of internal olefins compared to alpha olefins also may lead to formation of a considerably different product distribution. β-Sultones represent the primary reaction product initially obtained from sulfonation of internal olefins. The β-sultones obtained from internal olefins can be considerably more difficult to hydrolyze than are those obtained from alpha olefins, and they can sometimes be prone to desulfonation under typical hydrolysis conditions. In addition, the γ- and δ-sultones formed from rearrangement of β-sultones of internal olefins may be particularly resistant to hydrolysis. As a result, variable amounts of β-sultones and larger-ring sultones may sometimes persist after hydrolysis. Whereas alkenesulfonates bearing a sulfonate group on a terminal carbon atom are the primary reaction product formed from sulfonation of alpha olefins, hydroxysulfonates formed from hydrolysis of β-sultones are the primary sulfonation product formed from internal olefins. Like the hydroxysulfonates formed from alpha olefins, the hydroxysulfonates formed from internal olefins may similarly undergo dehydration to yield internal alkenesulfonates having variable positions for the double bond. Thus, like alpha olefins, sulfonation of internal olefins may produce a complex product distribution that may exhibit variable physical properties, such as surfactant properties, depending upon the actual product distribution obtained.

Although sultones may undergo hydrolysis to form hydroxysulfonates (or dehydration products formed therefrom) having useful surfactant properties, at least trace sultone quantities oftentimes remain following hydrolysis, particularly larger-ring sultones and sultones formed from internal olefins. Many sultones are skin sensitizers, and their continued presence in the mixture of reaction products following hydrolysis can lead to unsuitability for some surfactant applications, such as within medical or cosmetic compositions. In addition, some sultones undesirably inhibit foam formation in some surfactant applications. As a still further complicating factor, the reaction product mixture obtained from sulfonation of both alpha olefins and internal olefins oftentimes contains fairly substantial quantities of colored impurities, which may result in the material failing to pass color specifications for various types of applications. If the reaction product mixture fails to meet color specifications, costly and time-consuming bleaching operations may be performed in some cases. Bleaching can also result in partial revision to sultones in some cases.

Relevant publications include US 2014/290953; US 2015/284350; U.S. Pat. No. 4,715,991; and GB 1 340 977; as well as R. M. Shonk, et. al. “Reactions of Methylenecycloalkanes and Cycloalkylidenecycloalkanes with sulfur trioxide,” in 112 (7-8) RECUEIL DES TRAVAUX CHIMIQUES DES PAYS-BAS, 457 (1993); and C. Lalanne-Cassou, et. al., “Microemulsion formation with alkyl vinylidene sulfonates,” in 7(4) J. DISPERSION SCI. AND TECH. 479 (1986).

### SUMMARY

In any embodiment, the present disclosure provides methods for sulfonating vinylidene olefins preferably internal vinylidene olefins. The methods comprise: providing a vinylidene olefin comprising a vinylidene group; exposing the vinylidene olefin to a sulfonating reagent; and reacting the sulfonating reagent with the vinylidene olefin to form a sulfonated reaction product comprising at least one sulfonated compound formed from the vinylidene olefin.

In any embodiment, the present disclosure provides compositions comprising one or more sulfonated reaction products formed from vinylidene olefins. The compositions comprise: a sulfonated reaction product formed from a vinylidene olefin, in which the sulfonated reaction product comprises at least one sulfonated compound.

### BRIEF DESCRIPTION OF THE DRAWINGS

Not applicable.

### DETAILED DESCRIPTION

The present disclosure generally relates to surfactants and, more specifically, to sulfonated reaction products of vinylidene olefins having useful surfactant properties, and most preferably internal vinylidene olefins having such properties.

As discussed above, the sulfonated reaction products formed from alpha olefins and internal olefins may possess useful surfactant properties. These two classes of olefins may form different types and distribution ratios of sulfonated reaction products, which may impact their suitability for use in various surfactant applications. Innate structural differences and corresponding differences in the sulfonated reaction product distribution between these two classes of olefins may lead to distinct surfactant properties being obtained for each. The differing surfactant properties may lead to one class of sulfonated reaction products being preferable over the other for certain applications. For example, the sulfonated reaction products formed from internal olefins may be preferable to those obtained from alpha olefins for use in enhanced oil recovery (EOR) operations, Nevertheless, due to the availability of alpha olefins and their somewhat simpler reactivity pattern during sulfonation, the sulfonated reaction products of alpha olefins are in considerably wider use for surfactant applications. Difficulties precluding wider use of the sulfonated reaction products of both classes of olefins remain, however.

The present disclosure is directed to sulfonation of a class of olefins that differs structurally from both alpha olefins and internal olefins. Namely, the present disclosure to demonstrates that vinylidene olefins may be reacted to produce sulfonated reaction products under similar conditions to those used for sulfonating alpha olefins and internal olefins. As defined herein, vinylidene olefins bear a terminal alkene group in which C-2 of the alkene group is disubstituted with two hydrocarbyl groups and C-1 of the alkene group is unsubstituted and bears two hydrogen atoms. The generic structure of an internal vinylidene olefin suitable is for use in the present disclosure is shown in Formula 1 below.

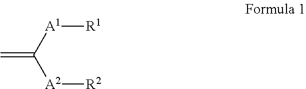

Preferably, the internal vinylidene olefin is a C₆ or C₈ to C₂₀ or C₂₆ or C₃₀ vinylidene olefin, meaning it has that number of carbon atoms. Collectively, A¹-R¹ and A²-R² define hydrocarbyl groups bound to C-2 of the alkene functionality. The variables are further defined hereinbelow, Thus, vinylidene olefins bear some extent of structural similarity to alpha olefins but are distinguished therefrom by the additional hydrocarbyl group at C-2, making it an “internal” alpha olefin or internal vinylidene. The presence of the additional hydrocarbyl group within vinylidene olefins may impact the reactivity profile observed during sulfonation and subsequent hydrolysis and lead to surprising surfactant properties, as discussed hereinafter. Without being limited by theory or mechanism, the hydrolysis of sultones formed during sulfonation of vinylidene olefins may occur at rates comparable to those of alpha olefin sultones, rather than at the slower rates of internal olefin sultones, since the olefinic bond is at a terminal location in both cases. Impurity profiles obtained during sulfonation of vinylidene olefins may also differ from those resulting from sulfonation of other classes of olefins.

Surprisingly, the sulfonated reaction products formed from vinylidine olefins are functionally distinguishable from those formed from alpha olefins and internal olefins having a like number of carbon atoms. In particular, the sulfonated reaction products formed from vinylidene olefins may exhibit a lower critical micelle concentration and C₂₀ values when dispersed in an aqueous fluid, particularly water, than do the corresponding sulfonated reaction products formed from alpha olefins and internal olefins. The lower critical micelle concentrations allow useful surfactant properties to be exhibited with smaller amounts of the sulfonated reaction products being used compared to the corresponding sulfonated reaction products of the other olefin types, potentially reducing the cost of surfactant materials for to various applications. Other physical properties of the sulfonated reaction products disclosed may be at least comparable to those obtained following sulfonation of alpha olefins and internal olefins. Because the sulfonated reaction products disclosed herein are structurally distinct from those obtained from other types of olefins, suitability for presently unmet surfactant applications may also be realized in some cases. The sulfonated reaction products disclosed herein may also be suitably used in any conventional application in which surfactant properties are desired.

As a still further advantage of the present disclosure, vinylidene olefins are a readily accessible class of compounds that may be obtained via dimerization of alpha olefins. Dimerization of alpha olefins to form vinylidene olefins may be promoted by numerous types of catalysts, including various metallocene catalysts. Alpha olefins having an even number of carbon atoms, in turn, may be readily synthesized through ethylene oligomerization using Ziegler-Matta catalysts or similar types of catalysts. Vinylidene olefins formed by dimerizing olefin mixtures having different olefin chain lengths, thereby forming a mixture of vinylidene olefins of differing carbon counts, may also be suitable and advantageous for use in the disclosure herein. Accordingly, vinylidene olefins represent a readily available and potentially inexpensive feedstock for producing sulfonated reaction products for use in commodity surfactant applications. Moreover, because olefin dimerization may be conducted with alpha olefins having a range of sizes (i.e., carbon counts), thereby affording a commensurate range of vinylidene olefin sizes and/or mixtures of sizes, an array of sulfonated reaction products having variable surfactant properties may be produced according to the disclosure herein. Additional tailoring of the surfactant properties may be realized by varying the reaction conditions to alter the distribution of sulfonated reaction products that are obtained following sulfonation according to the disclosure herein.

Unless otherwise indicated, room temperature is 25° C.

As used in the present disclosure and claims, the singular forms “a,” “an,” and “the” include plural forms unless the context clearly dictates otherwise.

The term “and/or” as used in a phrase such as “A and/or B” herein is intended to include “A and B,” “A or B,” “A”, and “B.”

For the purposes of the present disclosure, the new numbering scheme for groups of the Periodic Table is used. In said numbering scheme, the groups (columns) are numbered sequentially from left to right from 1 through 18, excluding the f-block elements (lanthanides and actinides).

The term “hydrocarbon” refers to a class of compounds containing hydrogen bound to to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different numbers of carbon atoms. The term “Cₙ” refers to hydrocarbon(s) or a hydrocarbyl group having n carbon atom(s) per molecule or group, wherein n is a positive integer. Such hydrocarbons or hydrocarbyl groups may be one or more of linear, branched, cyclic, acyclic, saturated, unsaturated, aliphatic, or aromatic.

The terms “saturated” or “saturated hydrocarbon” refer to a hydrocarbon or hydrocarbyl group in which all carbon atoms are bonded to four other atoms or bonded to three other atoms with one unfilled valence position thereon.

The terms “unsaturated” or “unsaturated hydrocarbon” refer to a hydrocarbon or hydrocarbyl group in which one or more carbon atoms are bonded to less than four other atoms, optionally with one unfilled valence position on the one or more carbon atoms.

The terms “hydrocarbyl” and “hydrocarbyl group” are used interchangeably herein. The term “hydrocarbyl group” refers to any C₁-C₁₀₀ hydrocarbon group bearing at least one unfilled valence position when removed from a parent compound. “Hydrocarbyl groups” may be optionally substituted, in which the term “optionally substituted” refers to replacement of at least one hydrogen atom or at least one carbon atom with a heteroatom or heteroatom functional group. Heteroatoms may include, but are not limited to, B, O, N, S, P, F, Cl, Br, I, Si, Pb, Ge, Sn, As, Sb, Se, and Te. Heteroatom functional groups that may be present in substituted hydrocarbyl groups include, but are not limited to, functional groups such as O, S, S═O, S(═O)₂, NO₂, F, Cl, Br, I, NR₂, OR, SeR, TeR, PR₂, AsR₂, SbR₂, SR, BR₂, SiR₃, GeR₃, SnR₃, PbR₃, where R is a hydrocarbyl group or H. Suitable hydrocarbyl groups may include alkyl, alkenyl, alkynyl, aryl, heteroaryl, cycloalkyl, cycloalkenyl, heterocyclyl, and the like, any of which may be optionally substituted.

The term “alkyl” refers to a hydrocarbyl group having no unsaturated carbon-carbon bonds, and which may be optionally substituted. The term “alkylene” refers to an alkyl group having at least two open valence positions.

The term “alkenyl” refers to a hydrocarbyl group having a carbon-carbon double bond, and which may be optionally substituted. The terms “alkene” and “olefin” are used synonymously herein. Similarly, the terms “alkenic” and “olefinic” are used synonymously herein. Unless otherwise noted, all possible geometric isomers are encompassed by these terms.

The terms “aromatic” and “aromatic hydrocarbon” refer to a hydrocarbon or to hydrocarbyl group having a cyclic arrangement of conjugated pi-electrons that satisfy the Hückel rule. The term “aryl” is equivalent to the term “aromatic” as defined herein. The term “aryl” refers to both aromatic compounds and heteroaromatic compounds, either of which may be optionally substituted. Both mononuclear and polynuclear aromatic compounds are encompassed by these terms.

Examples of saturated hydrocarbyl groups include, but are not limited to, methyl, ethyl, n-propyl, isopropyl, n-butyl, isobutyl, sec-butyl, tert-butyl, pentyl, isopentyl, hexyl, octyl, cyclopropyl, cyclobutyl, cyclopentyl, cyclohexyl, cyclooctyl, and the like, including their substituted analogues. Examples of unsaturated hydrocarbyl groups include, but are not limited to, ethenyl, propenyl, allyl, butadienyl, cyclopropenyl, cyclobutenyl, cyclopentenyl, cyclohexenyl, cyclooctenyl and the like, including their substituted analogues.

Examples of aromatic hydrocarbyl groups include, but are not limited to, phenyl, tolyl, xylyl, naphthyl, and the like, including all possible isomeric forms thereof. Polynuclear aromatic hydrocarbyl groups may include, but are not limited to, naphthalene, anthracene, indane, indene, and tetralin.

The term “vinylidene olefin” refers to an olefinic compound bearing a terminal double bond, in which C-1 is unsubstituted and C-2 is disubstituted with a first hydrocarbyl group and a second hydrocarbyl group, which may be the same or different. A double bond substituted as defined above may be referred to as a “vinylidene group. The structure of vinylidene olefins may be defined by Formula 1 above.

The terms “linear” and “linear hydrocarbon” refer to a hydrocarbon or hydrocarbyl group having a continuous carbon chain without side chain branching, in which the continuous carbon chain may be optionally substituted with heteroatoms or heteroatom groups.

The terms “branch,” “branched” and “branched hydrocarbon” refer to a hydrocarbon or hydrocarbyl group having a linear main carbon chain in which a hydrocarbyl side chain extends from the linear main carbon chain. Optional heteroatom substitution may be present in the linear main carbon chain or in the hydrocarbyl side chain.

The term “sulfonated reaction product” refers to one or more compounds formed directly or indirectly from interacting a sulfonating reagent with an olefinic compound, particularly a vinylidene olefin. It is to be appreciated that an elaborated sulfonate group may not necessarily be present initially in at least a portion of the sulfonated reaction product. Instead, the sulfonate group may be formed upon further reaction of an initial sulfonation product (e.g., through hydrolysis of one or more compounds, as described further herein).

Compositions of the present disclosure may comprise a sulfonated reaction product to formed from a vinylidene olefin, in which the sulfonated reaction product comprises at least one sulfonated compound. Vinylidene olefins that may be reacted with a sulfonating reagent according to the present disclosure may have a structure corresponding to Formula 1 (shown above). Mixtures of vinylidene olefins may undergo sulfonation in some cases.

Referring to Formula 1 above, A¹-R¹ and A²-R² each define a hydrocarbyl group that may be the same or different. When the vinylene olefin is prepared by dimerization of a single alpha olefin, as discussed further herein, the hydrocarbyl groups defined by A¹-R¹ and A²-R² differ from one another in carbon count by two carbon atoms. The two carbon atoms accounting for the differing carbon count are located within the vinylidene group of one of the olefin molecules undergoing dimerization. Vinylidene olefins prepared by dimerizing a mixture of alpha olefins having different chain lengths may feature hydrocarbyl groups with the same carbon count, although a mixture of vinylidene olefins may be produced in this case.

In more specific embodiments, the hydrocarbyl groups defined by A¹-R¹ and A²-R² bear a hydrogen atom at the allylic position with respect to the vinylidene group. Accordingly, A¹ and A² may independently be CH₂ or CHR³, wherein R³ is an optionally substituted, branched or unbranched alkyl group. For example, R³ may be selected from alkyl groups such as methyl, ethyl, n-propyl, isopropyl, n-butyl, isobutyl, sec-butyl, tert-butyl, pentyl, isopentyl, hexyl, octyl, cyclopropyl, cyclobutyl, cyclopentyl, cyclohexyl, cyclooctyl, and the like.

Referring still to Formula 1, R¹ and R² likewise may be the same or different and are selected from optionally substituted, branched or unbranched alkyl groups. As mentioned above, when a vinylidene olefin corresponding to Formula 1 is prepared by dimerization of a single alpha olefin, R¹ and R² differ from one another by two carbon atoms. In any embodiment, R¹ and R² may be independently selected from alkyl groups such as methyl, ethyl, n-propyl, isopropyl, n-butyl, isobutyl, sec-butyl, tert-butyl, pentyl, isopentyl, hexyl, octyl, cyclopropyl, cyclobutyl, cyclopentyl, cyclohexyl, cyclooctyl, and the like.

In any embodiment, R¹ and R² may be bonded together with one another to form a hydrocarbyl ring (carbocyclic ring), in which the vinylidene group is exocyclic to the ring. For example, In any embodiment, R¹ and R² may collectively be an ethylene group (i.e., —CH₂CH₂—) or a propylene group (—CH₂CH₂CH₂—).

Exposing a vinylidene olefin to a sulfonating reagent may produce a variety of sulfonated compounds, as outlined in Scheme 3 below. In Scheme 3, the groupings R^{1a}-A^{1a} and R^{2a}-A^{2a} correspond, respectively, to alkyl groups R¹ and R² defined above with respect to Formula 1. Thus, A^{1a} and A^{2a} may be CH₂ or CHR³, wherein R³ is defined in the same manner as above. Alkyl groups R¹ and R² have been redefined as R^{1a}-A^{1a} and R^{2a}-A^{2a} in Scheme 3 in order to show the various sultone rearrangement products that may form according to the present disclosure. Only one set of regioisomers is shown in Scheme 3 (those corresponding to the R^{2a}-A^{2a}-A² hydrocarbyl group), but it is to be appreciated that the other hydrocarbyl group may be reacted equivalently. Compositions of the present disclosure may comprise one regioisomer of the sulfonation products shown in Scheme 3, or mixtures of regioisomeric sulfonation products corresponding to those shown in Scheme 3 may be formed according to the disclosure herein. Similarly, the compositions of the present disclosure may be formed from mixtures of vinylidene olefins, thereby forming sulfonated reaction products having different chain lengths.

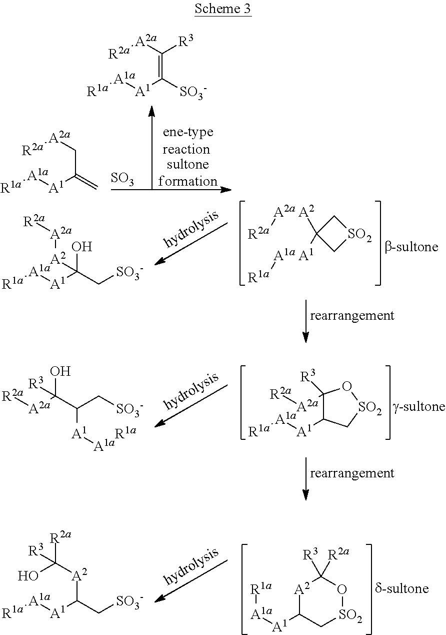

Thus, reaction of a vinylidene olefin with sulfur trioxide may form a sulfonated reaction product comprising an internal olefin bearing a terminal sulfonate group. Such sulfonated reaction products may be formed via an ene-type reaction. Similar internal olefins bearing a terminal sulfonate group may also be formed through dehydration of other sulfonated reaction products, as discussed hereinafter. The final product may be a Group 1 or Group 2 (of the Periodic Table) salt, such as a sodium or potassium salt.

The sulfonated reaction products formed from vinylidene olefins may also include one or more compounds that do not initially bear an elaborated sulfonate group. In particular, one or more sultones may be formed upon exposing a vinylidene olefin to sulfur trioxide or similar sulfonating reagents. The sultones may be converted into sulfonated reaction products through hydrolysis, specifically hydroxylated compounds (hydroxyalkanesulfonates) bearing a terminal sulfonate group, as discussed hereinafter.

Similar to the sulfonated reaction products formed from alpha olefins and internal olefins, the hydroxylated compounds formed upon hydrolysis of the corresponding sultones of vinylidene olefins may undergo dehydration to form one or more internal olefin sulfonates, in which the sulfonate group is present on a terminal carbon atom. The dehydration products are not shown in Scheme 3, but it is to be appreciated that one or more regioisomeric dehydration to products may be formed from each of the hydroxylated compounds. Since the sultones formed from vinylidene olefins each lead to hydroxylated compounds bearing a tertiary hydroxyl group, it is believed that dehydration may take place in some instances to form the corresponding internal olefins bearing a terminal sulfonate group. The possibility of the hydroxylated compounds formed from vinylidene olefins undergoing dehydration may represent a distinct advantage of conducting sulfonation reactions upon these types of olefin compounds. The dehydration may lead to greater product homogeneity (fewer products) and more consistent surfactant properties.

Accordingly, the sulfonated reaction products in the compositions disclosed herein may comprise an internal olefin bearing a terminal sulfonate group. Such sulfonated reaction products may be formed via an ene-type reaction of sulfur trioxide with the vinylidene group or as a dehydration product of a tertiary hydroxylated compound formed from a vinylidene olefin sultone. Preferably, the sulfonated reaction products may comprise at least one hydroxylated compound bearing a terminal sulfonate group, a dehydration product thereof, or any combination thereof. The at least one hydroxylated compounds may bear a tertiary hydroxyl group. Particular structure for the sulfonated reaction products are shown in Scheme 3.

Other sulfonated reaction products that may form according to the present disclosure include bis-sulfonate compounds. The bis-sulfonate compounds may be formed from any of the internal alkenesulfonates formed directly or indirectly from the vinylidene olefins, as discussed herein.

Compositions of the present disclosure may be formulated in solid form or dispersed in a suitable fluid phase. The fluid phase may comprise an aqueous fluid In any embodiment.

Accordingly, compositions of the present disclosure may further comprise an aqueous fluid in which the sulfonated reaction product is dissolved. Suitable aqueous fluids are not particularly limited and may be selected from deionized water, tap water, fresh water, surface water, ground water, brackish water, salt water, sea water, brine, or any combination thereof. Other aqueous fluid sources may also be suitable. The aqueous fluid may further comprise a water-miscible organic solvent such as alcohols, for example, In any embodiment.

When dissolved in a suitable aqueous fluid, the sulfonated reaction product may exhibit surfactant properties. According to some embodiments, the sulfonated reaction products may be present in the aqueous fluid above a critical micelle concentration. In any to embodiment, the sulfonated reaction products may exhibit a critical micelle concentration in water that is lower than that of an internal olefin sulfonate or an alpha olefin sulfonate having a corresponding number of carbon atoms.

The present disclosure also provides methods for synthesizing the sulfonated reaction products disclosed herein. The methods may comprise: providing a vinylidene olefin comprising a vinylidene group; exposing the vinylidene olefin to a sulfonating reagent; and reacting the sulfonating reagent with the vinylidene olefin to form a sulfonated reaction product comprising at least one sulfonated compound formed from the vinylidene olefin.

The sulfonating reagent may be sulfur trioxide in one or more embodiments of the present disclosure. A suitable ratio of sulfur trioxide to the vinylidene olefin may range from 1 to 1.2. By keeping the molar excess of sulfur trioxide relatively small, there is lower propensity for the initially formed internal olefin sulfonates to undergo a further reaction with excess sulfur trioxide to produce bis-sulfonate compounds. It is to be appreciated, however, that suitable ratios of sulfur trioxide to the vinylidene olefin that are larger or smaller than the foregoing ratio also fall within the scope of the present disclosure.

The vinylidene olefin may be exposed to the sulfur trioxide under thin film reaction conditions in a thin film reactor or, more specifically, falling film reaction conditions. Illustrative sulfonation conditions using falling film reaction conditions for alpha olefins are described in U.S. Pat. Nos. 3,169,142 and 4,052,431 and for internal olefins in U.S. Pat. No. 9,688,621. Further description of suitable sulfonation conditions for vinylidene olefins follows hereinafter.

Suitable falling film reaction conditions may comprise contacting a flowing thin film of the vinylidene olefin with a mixture of sulfur trioxide and a suitable diluent gas. The diluent gas may be non-reactive with the vinylidene olefin and be selected from among gases such as, for example, air, nitrogen, carbon dioxide, carbon monoxide, sulfur dioxide, halocarbon gases, methane, ethane, propane, butane, or any combination thereof. The mixture of sulfur trioxide and the diluent gas may promote turbulence in the flowing thin film to promote a reaction of the sulfur trioxide with the vinylidene olefin. Suitable ratios of the diluent gas to the sulfur trioxide may range between 5:1 to 50:1 by volume. In any embodiment, the sulfur trioxide and diluent gas may be heated (e.g., in a range of 40° C. to 50° C.) when combined with one another to reduce the likelihood of sulfur trioxide condensation.

The reaction between sulfur trioxide and the vinylidene olefin is exothermic. Active cooling of the flowing thin film may be conducted, for example, to decrease formation of colored byproducts when contacting the sulfur trioxide (i.e., during the reaction). Any suitable heat dissipation structure may be employed for removing heat from the flowing thin film.

The linear velocity of the sulfur trioxide and diluent gas may be modulated to regulate contact time of the sulfur trioxide with the flowing thin film and to produce a desired turbulence therein. In any embodiment, the mixture of sulfur trioxide and diluent gas may be flowed at a rate to produce a sulfur trioxide contact time of 1 second or less with the flowing thin film. Suitable back pressures of the mixture of sulfur trioxide and diluent gas may range from 4 to 7 pounds per square inch. The sulfur trioxide and diluent gas may be flowed concurrent or countercurrent with respect to the flow thin film. Further details concerning falling film and other thin film reaction conditions will be understood by one having ordinary skill in the art and are not described in further detail herein.

When the sulfonated reaction product initially comprises at least one sultone, methods of the present disclosure may comprise exposing the sulfonated reaction product to a hydroxide base, and reacting the hydroxide base with the at least one sultone to form at least one hydroxylated compound bearing a terminal sulfonate, a dehydration product thereof, or any combination thereof. The hydrolysis reaction of the at least one sultone with the hydroxide base may be conducted at room temperature or with heating. Room temperature reaction conditions may favor formation of the hydroxylated compounds bearing a terminal sulfonate group, whereas heating during hydrolysis may promote or favor dehydration to form one or more internal alkenes bearing a terminal sulfonate group. A mixture of internal alkenes bearing a terminal sulfonate group and hydroxylated compounds bearing a terminal sulfonate group may be obtained in some cases.

Suitable hydroxide bases may include, for example, alkali metal hydroxides, such as sodium hydroxide or potassium hydroxide. The amount of hydroxide base used with respect to the at least one sultone may be at least stoichiometric, and a molar excess is more typically used. Since δ-sultones are typically the slowest to hydrolyze, the hydrolysis reaction may be conducted until analyses indicate a substantially complete conversion of δ-sultones into another product. Following the completion of the hydrolysis reaction, any excess hydroxide base may be neutralized before further utilizing the reaction products described herein. The final product may be a Group 1 and/or Group 2 salt such as a sodium or potassium salt.

Further embodiments of the present disclosure may comprise bleaching the sulfonated reaction product. Bleaching may be conducted, for example, if the sulfonated reaction product does not meet color specifications for an intended application. Bleaching of the sulfonated reaction product may comprise contacting the sulfonated reaction product with an aqueous sodium hypochlorite solution for a sufficient length of time to reach a desired color specification. Hydrogen peroxide may be used as an alternative bleaching agent. Suitable to bleaching times may range from 5 minutes to 8 hours. A temperature of 25° C. to 60° C. may be employed for the bleaching operation. The pH may be maintained at a level of 9 or greater to discourage reformation of sultone(s) during the bleaching process.

Methods of the present disclosure may also comprise preparing the vinylidene olefin to undergo sulfonation according to the disclosure herein. A number of techniques are available for preparing vinylidene olefins suitable for use in the disclosure herein, and particular methods for forming the vinylidene olefins are not considered to be especially limited. Illustrative methods for forming vinylidene olefins by dimerizing alpha olefins may be found in U.S. Pat. Nos. 4,658,078, 4,973,788, and 5,625,105.

Vinylidene olefins corresponding to Formula 1 may be synthesized by dimerizing an alpha olefin in the presence of a suitable dimerization catalyst. Dimerization may take place via sequential 1,2-metal additions to form the vinylidene olefin. Suitable alpha olefins may be branched or unbranched and may be C₆ to C₁₂ alpha olefins, according to more particular embodiments. Accordingly, the vinylidene olefins resulting from dimerization of such alpha olefins may have a carbon count of C₁₂ to C₂₄ before undergoing sulfonation according to the disclosure herein. The vinylidene olefins may have a structure corresponding to Formula 1, in which the hydrocarbyl groups differ from one another by two carbon atoms. Mixtures of alpha olefins may also be dimerized under similar conditions to afford mixtures of vinylidene olefins.

Suitable dimerization catalysts for converting alpha olefins into vinylidene olefins may be metallocene catalysts, In any embodiment. In any embodiment, suitable metallocene catalysts may comprise a cyclopentadienyl zirconium or hafnium metallocene. An alumoxane co-catalyst may also be present in such catalyst systems. The atomic ratio of Zr or Hf to Al may range from 1:1 to 1:100. Ziegler-type catalysts comprising trialkylaluminum compounds may also comprise suitable catalysts In any embodiment. Suitable catalytic conditions employing trialkylaluminum compounds may include, for example, providing 0.001-0.05 equivalents of the trialkylaluminum compound with respect to the alpha olefin and heating at a temperature from 100° C. to 170° C.

Embodiments disclosed herein include:

A. Methods for sulfonating vinylidene olefins. The methods comprise: providing a vinylidene olefin comprising a vinylidene group; exposing the vinylidene olefin to a sulfonating reagent; and reacting the sulfonating reagent with the vinylidene olefin to form a sulfonated reaction product comprising at least one sulfonated compound formed from the vinylidene olefin.

B. Compositions comprising one or more sulfonated reaction products of to vinylidene olefins. The compositions comprise a sulfonated reaction product formed from a vinylidene olefin, the sulfonated reaction product comprising at least one sulfonated compound.

Embodiments A and B may have one or more of the following additional elements in any combination:

Element 1: wherein the sulfonating reagent comprises sulfur trioxide.

Element 2: wherein a molar ratio of the sulfonating reagent to the vinylidene olefin ranges from 1 to 1.2.

Element 3: wherein the vinylidene olefin is exposed to the sulfur trioxide under thin film reaction conditions or falling film reaction conditions.

Element 4: wherein at least a portion of the sulfonated reaction product comprises an internal olefin bearing a terminal sulfonate group.

Element 5: wherein at least a portion of the sulfonated reaction product initially comprises at least one sultone, the method further comprising: exposing the sulfonated reaction product to a hydroxide base; and reacting the hydroxide base with at least a portion of the at least one sultone to form at least one hydroxylated compound bearing a terminal sulfonate group, a dehydration product thereof, or any combination thereof.

Element 6: wherein the at least one sultone comprises at least a β-sultone.

Element 7: wherein at least a portion of the sulfonated reaction product comprises a bis-sulfonate compound.

Element 8: wherein the vinylidene olefin is prepared by dimerizing an alpha olefin or a mixture thereof.

Element 9: wherein the alpha olefin is a C₆ to C₁₂ alpha olefin or a mixture thereof.

Element 10: wherein the alpha olefin is dimerized by exposing the alpha olefin to a dimerization catalyst.

Element 11: wherein the dimerization catalyst comprises a metallocene.

Element 12: wherein the vinylidene olefin, especially internal vinylidene olefin, has a structure of

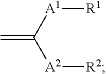

wherein R¹ and R² are optionally substituted, branched or unbranched alkyl groups, R¹ and R² being the same or different; and wherein A¹ and A² are independently CH₂ or CHR³; wherein R³ is an optionally substituted, branched or unbranched alkyl group; wherein preferably, the internal vinylidene olefin is a C₆ or C₈ to C₂₀ or C₂₆ or C₃₀ vinylidene olefin, meaning it has that number of carbon atoms.

Element 13: wherein at least a portion of the sulfonated reaction product comprises at least one hydroxylated compound bearing a terminal sulfonate group, a dehydration product thereof, or any combination thereof.

Element 14: wherein the composition further comprises: an aqueous fluid in which the sulfonated reaction product is dissolved.

Element 15: wherein the sulfonated reaction product is present in the aqueous fluid above a critical micelle concentration.

Element 16: wherein the sulfonated reaction product has a lower critical micelle concentration in water than does a sulfonated reaction product formed from an internal olefin having a like number of carbon atoms.

By way of non-limiting example, exemplary combinations applicable to A include:

The method of A in combination with elements: 1 and 2; 1 and 3; 1-3; 1 and 4; 1 and 5; 1 and 7; 1 and 8; 1 and 12; 1, 5 and 9; 2 and 3; 2 and 4; 2 and 5; 2 and 7; 2 and 8; 2 and 12; 2, 8 and 9; 3 and 4; 3 and 5; 3 and 7; 3 and 8; 3 and 12; 3, 8 and 9; 4 and 5; 4 and 8; 4 and 12; 4, 8 and 9; 5 and 6; 5 and 7; 5 and 8; 5 and 12; 5, 8 and 9; 7 and 8; 7 and 12; and 7-9.

By way of non-limiting example, exemplary combinations applicable to B include:

The method of B in combination with elements: 4 and 7; 4 and 12; 4 and 13; 4 and 14; 4 and 15; 4 and 16; 7 and 12; 7 and 13; 7 and 14; 7 and 15; 7 and 16; 12 and 13; 12 and 14; 12 and 15; 12 and 16; 4, 7 and 12; 4, 7 and 13; 4, 7 and 14; 4, 7 and 15; 4, 7 and 16; 4, 7, 12 and 13; 4, 7, 12 and 14; 4, 7, 12 and 15; 4, 7, 12 and 16; 13 and 14; 13 and 15; 13 and 16; 14 and 15; 14 and 16; and 15 and 16.

To facilitate a better understanding of the embodiments described herein, the following examples of various representative embodiments are given. In no way should the following examples be read to limit, or to define, the scope of the present disclosure.

### EXAMPLES

1-Decene and 1-octene were separately dimerized using a metallocene catalyst to afford the corresponding vinylidene olefins having 20 and 16 carbon atoms, respectively. The respective vinylidene olefin product structures are shown in Formulas 2 and 3 below. Prior to sulfonation, the two vinylidene olefins were purified by distillation to remove trimers and heavier oligomers, preferably isolating internal vinylidene olefins.

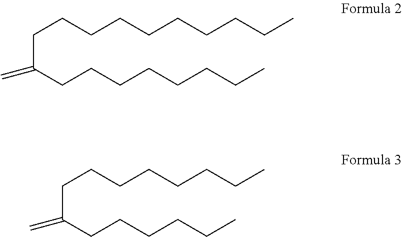

Example 1: The vinylidene olefins of Formulas 2 and 3 were separately reacted with sulfur trioxide in a thin film reactor, such as under the conditions described in U.S. Pat. No. 3,169,142, to produce a mixture of reaction products, primarily the corresponding alkene sulfonic acids and sultones. More specifically, the reaction was conducted in a continuous thin film reactor at a temperature of 120° F. and a vinylidene olefin feed rate of 200 g/hr. Sulfur dioxide and air were combined to form sulfur trioxide at flow rates and ratios sufficient to afford a sulfur trioxide to vinylidene olefin ratio of 1.2:1 on a molar basis, with excess air being present as a diluent. The back pressure was maintained at 12-20 psi. After sulfonation, the mixture of reaction products was combined with aqueous sodium hydroxide to convert the sultones to hydroxylated compounds or their corresponding dehydration products. In particular, the mixture of reaction products was dripped into a stirred metal container holding a slight excess of sodium hydroxide in water relative to the molar amount of sulfur trioxide used. The amount of water used was sufficient to target a final activity of 25 wt. % olefin sulfonate (37.5 g of 50% NaOH and 287.5 g of water per 100 g of collected reaction product). After combining, the solution was transferred to a glass round bottom flask equipped with a thermocouple, stirrer, and heating mantle. The solution was heated to boiling for at least 30 minutes until the two-phase activity remained unchanged. The pH was monitored during the heating process, and a few drops of 50% NaOH were added if the pH reached 7 at any time.

Comparative Sample 1: A comparative sample was prepared by sulfonating a mixture of C₁₆ internal olefins under sulfonation conditions similar to those described in Example 1. The C₁₆ internal olefins were prepared by acid-catalyzed dimerization of 1-octene, which places the double bond at variable internal locations within the hydrocarbon chain.

Comparative Sample 2: A commercial sample of sulfonated C₁₂ alpha olefin having 45% activity (Pilot Chemical Calsoft AOS C12-45) was also obtained for further testing, as provided below.

Example 2: Surfactant Properties. Table 1 below summarizes the surfactant properties for the two sulfonated reaction products from Example 1 (Entries 1 and 2, C₂₀ and C₁₆ vinylidene olefin starting materials, respectively), the sulfonated reaction products of Comparative Sample 1 (Entry 3, internal olefin starting materials), and the sulfonated reaction products of Comparative Sample 2 (Entry 4, alpha olefin starting material). Surfactant activities were measured using conditions specified in ASTM D3049. The percent active material for Entries 1 and 2 after sulfonation and hydrolysis were 13.1% and 14.5%, respectively. Critical micelle concentrations in water and 1% NaCl solution, surface tension values at the critical micelle concentration, and C₂₀ values were measured using conditions specified in ISO 4311. Foaming properties were measured using conditions specified in ASTM D1173-07. Wetting was measured using conditions specified in ASTM D2281. Interfacial tension (IFT) values were measured using conditions specified in ASTM D1331. C₂₀ values represent the surfactant concentration needed to decrease the surface tension of the solvent by 20 mN/m.

Calcium tolerance was tested using the following procedure. A 0.1 wt. % solution was prepared by dissolving 0.050 g of the sulfonated reaction product in 50 mL of distilled water in a 200 mL Erlenmeyer flask. This solution was used as a blank to set a turbidity value of 0 on a LaMotte 2020 Turbidity Meter. A 1.00 wt. % solution of calcium chloride was titrated into the sulfonated reaction product solution in 0.20 mL increments using a 5 mL micro-buret. The solution was then mixed and the turbidity was read again after each calcium chloride aliquot addition. The haze reading was then plotted against the titer (volume of added calcium chloride solution). The amount of added calcium chloride solution needed to produce a haze reading of 50 was then determined. This reading represents the lowest perceptible haze. The titer volume and concentration and the sample concentration may then be used to determine the number of milligrams of calcium that may be tolerated per gram of sample before haziness occurs. That is, the calcium tolerance values presented herein represent the amount of calcium that may be present per gram of sample before precipitation of the surfactant occurs.

TABLE 1

| | Foaming of | | | | | | |
|---|---|---|---|---|---|---|---|
| | 0.1 Wt. % | | Wetting of | Calcium | | CMC | |
| | surfactant | | 0.1 Wt. % | Tolerance | | | 1% NaCl |
| | Initial | 5 min. | surfactant | (mg Ca/g | IFT | DI H₂O | soln. |
| Entry | (mm) | (mm) | (sec) | sample) | (mN/m) | (wt. %) | (wt. %) |
| 1 | 100 | 92 | 89 | 60 | 2.94 | 0.005 | 0.017 |
| 2 | 162 | 150 | 3.2 | 31 | 0.5 | 0.007 | 0.014 |
| 3 | 150 | 145 | 5.8 | 37 | 0.51 | 0.4 | 0.016 |
| 4 | 130 | 124 | 80 | 276 | 0.26 | 0.14 | 0.05 |

| | | | C₂₀ | |
|---|---|---|---|---|
| | ST | | | 1% NaCl |
| | DI H₂O | 1% NaCl | DI H₂O | soln. |
| Entry | (nM/m) | (nM/m) | (wt. %) | (wt. %) |
| 1 | 29 | 25 | 0.000013 | 0.00017 |
| 2 | 33 | 24 | 0.00021 | 0.00012 |
| 3 | 28 | 26 | 0.0015 | 0.00013 |
| 4 | 33 | 32 | 0.018 | 0.005 |

As shown in Table 1, both sulfonated reaction products of vinylidene olefins (Entries 1 and 2) exhibited a critical micelle concentration in water at considerably lower concentrations than did the sulfonated reaction products of an internal olefin mixture (Entry 3) to or an alpha olefin (Entry 4). The critical micelle concentrations were higher and closer to one another in 1% NaCl solution. The C₂₀ values for the sulfonated reaction products of vinylidene olefins in deionized water also occurred at much lower concentrations than did the sulfonated reaction products of either the internal olefin mixture or the alpha olefin.

As also shown in Table 1, there was significant variance between the sulfonated reaction products of vinylidene olefins depending on the number of carbon atoms present in the reaction product. For example, the C₂₀ sulfonated reaction product (Entry 1) exhibited a higher wetting value and greater calcium tolerance than did the corresponding C₁₆ sulfonated reaction product (Entry 2). The C₁₆ sulfonated reaction product (Entry 2) was comparable to the sulfonated reaction product of the internal olefin mixture (Entry 3) with respect to these properties. In contrast, the C₂₀ sulfonated reaction product (Entry 1) exhibited a wetting value more similar to that of the sulfonated reaction product of an alpha olefin (Entry 4).

As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the to compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Whenever a numerical range with a lower limit and an upper limit is disclosed, any number and any included range falling within the range is specifically disclosed. In particular, every range of values (of the form, “from a to b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

What is claimed is:

1. A method comprising:
providing an internal vinylidene olefin comprising a vinylidene group;
exposing the vinylidene olefin to a sulfonating reagent; and
reacting the sulfonating reagent with the internal vinylidene olefin to form a sulfonated reaction product comprising at least one sulfonated compound formed from the vinylidene olefin;
wherein the internal vinylidene olefin is a C₆ to C₃₀ vinylidene olefin and has a structure of 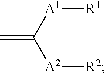
wherein R¹ and R² are optionally substituted, branched or unbranched alkyl groups, R¹ and R² being the same or different; and
wherein A¹ and A² are independently CH₂ or CHR³;
wherein R³ is an optionally substituted, branched or unbranched alkyl group.

2. The method of claim 1, wherein the sulfonating reagent comprises sulfur trioxide.

3. The method of claim 1, wherein a molar ratio of the sulfonating reagent to the vinylidene olefin ranges from 1 to 1.2.

4. The method of claim 2, wherein the internal vinylidene olefin is exposed to the sulfur trioxide under thin film reaction conditions or falling film reaction conditions.

5. The method of claim 1, wherein at least a portion of the sulfonated reaction product comprises an internal olefin bearing a terminal sulfonate group.

6. The method of claim 1, wherein at least a portion of the sulfonated reaction product initially comprises at least one sultone, the method further comprising:
a) exposing the sulfonated reaction product to a hydroxide base; and
b) reacting the hydroxide base with at least a portion of the at least one sultone to form at least one hydroxylated compound bearing a terminal sulfonate group, a dehydration product thereof, or any combination thereof.

7. The method of claim 6, wherein the at least one sultone comprises at least a β-sultone.

8. The method of claim 1, wherein at least a portion of the sulfonated reaction product comprises a bis-sulfonate compound.

9. The method of claim 1, wherein the internal vinylidene olefin is prepared by dimerizing an alpha olefin or a mixture thereof.

10. The method of claim 9, wherein the alpha olefin is a C₆ to C₁₂ alpha olefin or a mixture thereof.

11. The method of claim 9, wherein the alpha olefin is dimerized by exposing the alpha olefin to a dimerization catalyst.

12. The method of claim 11, wherein the dimerization catalyst comprises a metallocene.

13. A composition comprising a sulfonated reaction product formed from an internal vinylidene olefin, the sulfonated reaction product comprising at least one sulfonated compound; wherein at least a portion of the sulfonated reaction product initially comprises at least one sultone; wherein the internal vinylidene olefin is a C₆ to C₃₀ vinylidene olefin and has a structure of 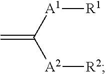
wherein R¹ and R² are optionally substituted, branched or unbranched alkyl groups, R¹ and R² being the same or different; and
wherein A¹ and A² are independently CH₂ or CHR³;
wherein R³ is an optionally substituted, branched or unbranched alkyl group.

14. The composition of claim 13, wherein at least a portion of the sulfonated reaction product comprises an internal olefin bearing a terminal sulfonate group.

15. The composition of claim 13, wherein at least a portion of the sulfonated reaction product comprises at least one hydroxylated compound bearing a terminal sulfonate group, a dehydration product thereof, or any combination thereof.

16. The composition of claim 13, wherein at least a portion of the sulfonated reaction product comprises a bis-sulfonate compound.

17. The composition of claim 13, further comprising:
an aqueous fluid in which the sulfonated reaction product is dissolved.

18. The composition of claim 17, wherein the sulfonated reaction product is present in the aqueous fluid above a critical micelle concentration.

19. The composition of claim 18, wherein the sulfonated reaction product has a lower critical micelle concentration in water than does a sulfonated reaction product formed from an internal olefin having a like number of carbon atoms.
